(19) United States
(12) Patent Application Publication
(10) Pub. No.: US 2024/0141328 A1
(43) Pub. Date: May 2, 2024

(54) Assay for Massive Parallel RNA Function Perturbation Profiling

(71) Applicant: Ladder Therapeutics, Inc., Portland, OR (US)

(72) Inventor: Rabia Khan, Toronto (CA)

(73) Assignee: Ladder Therapeutics, Inc., Portland, OR (US)

(21) Appl. No.: 18/549,340

(22) PCT Filed: Mar. 8, 2021
(86) PCT No.: PCT/EP2022/055951

§ 371 Date: Sep. 6, 2023

(30) Foreign Application Priority Data
Mar. 8, 2021 (GB) 2103216.4

(51) Int. Cl. C12N 15/10; G01N 21/64; G01N 33/50

(52) U.S. Cl. CPC C12N 15/1072; G01N 21/6486; G01N 33/5023; C12N 2310/531; C12N 2840/203

(57) ABSTRACT

This invention features nucleic acid constructs which comprises reporter genes and a query sequence, wherein the query sequences encode or are RNA folded into a secondary structure and or RNA regulatory elements. These nucleic acid constructs can be used in massively parallel assay methods for perturbation profiling also disclosed herein. Such methods provide the ability to study the effect of chemical or genetic perturbations to modulate RNA within an intracellular context.

### FIELD OF THE INVENTION

The present invention relates to screening methods involving panels of cells comprising RNA constructs and particularly, although not exclusively, to their use in a cell-based screening platform.

### BACKGROUND

It is well established that RNA is an attractive target drug discovery and development, both to drug so-called “undruggable” proteins, and as a mechanism to regulate the non-coding genome. However, the ability to modulate RNA with small molecules has yet to be achieved in a systematic way.

Previous research has demonstrated that RNA forms secondary and tertiary structures within a cellular context. These RNA structural motifs are critical across a diverse range of biological functions such as regulation of gene expression, RNA splicing, and translation. These function within a cell as a result of functional interactions with other cellular constituents such as RNA-protein interactions (RNA interacts with RNA Binding proteins) and RNA-DNA interactions (RNA binding to DNA), to name a few.

There are a number of biochemical assays to screen small-molecules against RNA structures (for example microarray, ASMS, Alphascreen) which allow for a target-by-target screening approach. Although these methods allow for large chemical libraries to be screened against an RNA target, the lack of a cellular context remains a challenge as the native cellular-RNA functions and structures are not considered in these methods.

Cellular systems to screen small-molecules that bind to RNA structures within the cellular context have also been developed, on a target-by-target basis. Although the native RNA structure is maintained within a cellular context, high throughput screening across multiple targets using this method is laborious and requires the building of multiple chemical libraries.

The existing methods require the identification of a single target and cannot study the effect of a large number of molecules on a large number of RNA structures. Until now, the ability to study the effect of chemical or genetic perturbations to modulate RNA within an intracellular context, has yet to be achieved in a systematic way.

### SUMMARY OF THE INVENTION

The inventors have identified large numbers of RNA secondary structures in the genome, using in silico methods. For instance, around 4,000 G-quadruplex structures were identified. The present invention firstly enables the functional nature of these structures within their endogenous genomic context, such as within a UTR, and this function to be probed and elucidated, e.g. within a cell. This form of screen opens-up new fields of biology for investigation as it allows the study of the effect of chemical or genetic perturbations to RNA structures at scale, within a native biological context. Secondly, the present invention provides the means—e.g. by using a library of cells containing functional constructs comprising these secondary structures—to screen for candidate agents (e.g. therapeutic agents, small molecules, further RNA molecules) that can interact with these newly identified structures. This can be termed ‘multiplexed RNA structure small molecule screening’. The ability of e.g. small molecules to specifically and selectively bind the RNA structural motifs and disrupt a biological functional within a cell makes it possible to target novel biology, previously targeted by classical drug discovery.

Accordingly, in a first aspect, this invention provides a nucleic acid construct comprising i.) a first sequence that encodes a first reporter gene; and ii.) a second sequence that encodes a second reporter gene and comprises a query sequence, wherein the query sequence is operably linked to the second reporter gene and wherein the query sequence encodes or is: an RNA folded into a secondary structure and/or an RNA regulatory element of a gene transcript, wherein said secondary structure and/or transcript is not part of the transcript of the second reporter gene. The nucleic acid construct also comprises one or more promoters capable of driving the expression of the first and second sequences.

In some embodiments, the second sequence comprises a barcode sequence that is unique to the query sequence.

The one or more promoters (iii) may comprise a first promoter capable of driving the expression of the first sequence; and a second promoter capable of driving the expression of the second sequence (wherein the first and second promoters can operate independently of each other). In other embodiments, the nucleic acid construct comprises a single promoter capable of driving the expression of the first and second sequences. The single promoter may be a bidirectional promoter or a unidirectional promoter.

In preferred embodiments, the query sequence encodes, or is, an RNA regulatory element that is an untranslated region (UTR). The UTR is from a heterologous gene (that is, the UTR of a gene that is not the second reporter gene.) In some embodiments, the UTR is a 5′ UTR of a heterologous gene. In other embodiments, the UTR is a 3′ UTR of a heterologous gene. The UTR in the query sequence may be mutated with respect to the UTR sequence of a native gene. For instance, the UTR in the query sequence may comprise 1, 2, 3, 4, 5, 6, 7, 8, 9, 10 or more single nucleotide deletions, substitutions and/or insertions.

The UTR may comprise one or more RNA secondary structures, e.g. a G-quadruplex (G4), a triple helix, a pseudoknot, a stem-loop, and/or a multiway junction. The UTR may contain an internal ribosome entry site (IRES).

In some embodiments, the query sequence encodes, or is, an RNA regulatory element that is an intron.

In some embodiments, the query sequence encodes, or is, an RNA regulatory element that is an internal ribosome entry site (IRES).

The intron or IRES may comprise one or more RNA secondary structures, e.g. a G-quadruplex (G4), a triple helix, a pseudoknot, a stem-loop, and/or a multiway junction.

In some embodiments, the nucleic acid construct comprises a bicistronic reporter system wherein the first and second reporter genes are disposed with the IRES therebetween.

In some embodiments, the query sequence encodes, or is, a secondary structure comprising a G-quadruplex (G4). In some embodiments, the query sequence encodes, or is, a secondary structure comprising a triple helix. In some embodiments, the query sequence encodes, or is, a secondary structure comprising a pseudoknot. In some embodiments, the query sequence encodes, or is, a secondary structure comprising a stem-loop. In some embodiments, the query sequence encodes, or is, a secondary structure comprising a multiway junction.

The query sequence may encode, or comprise, a wild type RNA that includes a portion that is folded into a secondary structure. Alternatively, the query sequence may encode, or comprise, a mutant RNA, wherein the mutant RNA comprises one or more mutations relative to a wild type sequence of an RNA that includes a portion that is folded into a secondary structure. These secondary structures may comprise a G-quadruplex (G4), a triple helix, a pseudoknot, a stem-loop, and/or a multiway junction. In some embodiments, the mutations relative to a wild type sequence are known to be, or are suspected of being, mutations associated with a biological disease state.

In some embodiments, the query sequence encodes or is a secondary structure comprising an RNA folded into a secondary structure from a class of coding or non-coding RNA sequences expressed by an organism of interest, wherein the class of non-coding RNA sequences is selected from introns or UTRs of mRNA, lncRNA, miroRNA or cirRNA.

In some embodiments, the query sequence encodes or comprises two or more secondary structures selected from the list consisting of G4, pseudoknot, stem loop, triple helix, and multiway junction, or functional units such as a UTR or an IRES. The two or more secondary structures may form a functional unit such as a UTR or an IRES.

A stem loop is an RNA structure comprising base pairing between two regions of the same single strand of RNA which ends in an unpaired loop. Stem loops are also known as hairpins or hairpin loops, in particular when the unpaired loop is short.

In some embodiments, the two or more secondary structures form a functional unit such as a UTR. For instance, the UTR may comprise an IRES and another secondary structure.

In some embodiments, the first and/or second reporter genes express fluorescent proteins such as green fluorescent protein (GFP), red fluorescent protein (RFP), yellow fluorescent protein (YFP), orange fluorescent protein (OFP), and/or blue fluorescent protein (BFP). Other suitable reporter genes include luciferase. In some embodiments, one of the first and second reporter genes is green fluorescent protein (GFP). The GFP may be a destabilised GFP. Similarly, the RFP, YFP, OFP and/or BFP may be destabilised.

In some embodiments herein, the second reporter gene may be termed a ‘first domain’ and the query sequence may be termed a ‘second domain’.

The invention also provides a vector comprising the nucleic acid construct of any one of the preceding claims. The vector may comprise DNA or RNA. The vector may be a viral or a non-viral vector. In some embodiments, the viral vector is a lentiviral vector. Libraries of these vectors are also provided.

In some embodiments the vector may comprise a selectable marker such as a resistance gene. Such selectable markers can assist in the cloning and selection of the vector and/or the selection of cells containing the vector. The selection marker can be Puromycin, Hygromycin, Neomycin or Blasticidin.

The invention also provides a library of cells, comprising a multiplicity of cell populations each comprising one or more cells comprising a nucleic acid construct according to the first aspect of the invention, wherein each nucleic acid construct of the cells of one single population are the same as each other, but wherein each nucleic acid construct of cells of different populations are different from each other. In some aspects, the library of cells comprise a multiplicity of cell populations each comprising one or more cells comprising an RNA construct, wherein each RNA construct of the cells of one single population are the same as each other, but wherein the RNA constructs of cells of different populations are different from each other; wherein each RNA construct comprises a first domain comprising an expression cassette capable of expressing a reporter gene, and a second domain in which the RNA is folded into a secondary structure or a combination of structures and/or comprises an RNA regulatory element of a gene transcript that is not the reporter gene transcript.

In a second aspect, this invention provides a library of RNA constructs, wherein each RNA construct comprises a first domain comprising an expression cassette capable of expressing a reporter gene, and a second domain in which the RNA is folded into a secondary structure and/or comprises an RNA regulatory element of a gene transcript that is not the reporter gene transcript.

In a third aspect, this invention provides a library of vectors comprising or encoding the library of RNA constructs according to the second aspect of the invention. The vectors may be viral vectors or non-viral vectors. In some embodiments in which the vectors of the library are non-viral vectors, they may comprise a DNA plasmid that expresses the RNA construct. In other embodiments in which the vectors of the library are non-viral vectors, they may comprise the RNA construct itself. In embodiments in which the vectors of the library are viral vectors, they may be lentiviral vectors, e.g. integrating lentiviral vectors. Other viral vectors are well known to the skilled person, as disclosed herein, and may be employed in the working of this invention.

In a fourth aspect, this invention provides multiplexed methods of screening a panel of candidate agents to select agents that interact with an RNA regulatory element and/or secondary structure, the method comprising:
a. contacting the library of cells according to the first aspect with the panel of candidate agents in a multiplexed fashion, then
b. identifying (a) cell population(s) in which reporter gene expression is increased or decreased relative to the average reporter gene expression, then
c. selecting the candidate agents that had been contacted with said cell population(s) identified in step b.

In a fifth aspect, this invention provides methods of screening a population of RNA regulatory elements and/or secondary structures to assess their function. In this aspect, the method comprises taking a library of cells according to the first aspect, propagating the cells, and assessing the expression level of the reporter gene in each cell population. In line with the constructs of the other aspects of the invention, each RNA construct of the cells of one single population are the same as each other, but wherein the RNA constructs of cells of different populations are different from each other. Each RNA construct comprises a first domain comprising an expression cassette capable of expressing a reporter gene, and a second domain in which the RNA is folded into a secondary structure and/or comprises an RNA regulatory element of a gene transcript that is not the reporter gene transcript.

In some embodiments, the RNA construct is stably expressed by the cells. In other embodiments, the RNA construct is transiently expressed by the cells. The cells may have been genetically modified to knock-out, knock-down, or silence one or more RNA-binding proteins. The cells may be patient derived cells, or be iPS derived cells. In some embodiments, the cell populations are pooled within a single culture volume. In other embodiments, each cell populations in a cell culture volume that is separate from the respective cell culture volumes of each other cell population.

The cells used in the libraries (and methods) of this invention may be eukaryotic cells, mammalian cells, for instance human cells, cell lines, primary cells, e.g. those taken from healthy or diseased individuals. The cells may comprise further genetic modifications such as genetic knock-outs, e.g. as described herein.

In preferred embodiments, each RNA construct comprises a barcode sequence. Each barcode sequence is unique to each second domain. Thus, reading the barcode (e.g. via sequencing, etc) enables the species of the second domain to be determined.

In some embodiments, the combination of RNA structures can affect translation of the mRNA, or splicing of the mRNA through binding to RNA Binding Proteins. The library may comprise UTR-containing multiple RNA constructs that comprise wildtype UTR with a combination of RNA structures as well as RNA structures that carry mutations disrupting the RNA structure; and wherein other cell populations of the library comprise UTR-containing RNA constructs that comprise mutations in the RNA structures. The RNA constructs in the cells may constitute a panel comprising UTR structures from a class of coding or non-coding RNA sequences expressed by an organism of interest (e.g. human), wherein the class of non-coding RNA sequences is selected from mRNA UTRs, lncRNAs, miroRNAs and cirRNAs. The RNA constructs in the cells may comprise a large proportion, or substantially all of, the UTR structures expressed by the genome of an organism of interest, for instance at least 50%, at least 60%, at least 70%, at least 75%, at least 80%, at least 85%, at least 90% or at least 95% of these structures, e.g. as defined by in silico or in vitro screens.

Similarly, the library may comprise stemloop- or multiway junction-containing multiple RNA constructs that comprise wildtype stemloops or multiway junctions with a combination of RNA structures as well as RNA structures that carry mutations disrupting the RNA structure; and wherein other cell populations of the library comprise stemloop- or multiway junction-containing RNA constructs that comprise mutations in the RNA structures. The RNA constructs in the cells may constitute a panel comprising stemloop or multiway junction structures from a class of coding or non-coding RNA sequences expressed by an organism of interest (e.g. human), wherein the class of non-coding RNA sequences is selected from mRNA UTRs, lncRNAs, miroRNAs and cirRNAs. The RNA constructs in the cells may comprise a large proportion, or substantially all of, the stemloop or multiway junction structures expressed by the genome of an organism of interest, for instance at least 50%, at least 60%, at least 70%, at least 75%, at least 80%, at least 85%, at least 90% or at least 95% of these structures, e.g. as defined by in silico or in vitro screens

In some embodiments, the combination of RNA structures can form a functional domain, such as an IRES (internal ribosomal entry site). This second domain may comprise a combination of secondary structures that collectively comprise an internal ribosome entry site (IRES). The library may comprise IRES-containing RNA constructs that comprise wildtype IRES structure(s); and wherein other cell populations of the library comprise IRES-containing RNA constructs that comprise mutant IRES structures. The RNA constructs in the cells may constitute a panel comprising IRES structures from a class of coding or non-coding RNA sequences expressed by an organism of interest (e.g. human), wherein the class of non-coding RNA sequences is selected from mRNA UTRs, lncRNAs, miroRNAs and cirRNAs. The RNA constructs in the cells may comprise a large proportion, or substantially all of, the IRES structures expressed by the genome of an organism of interest, for instance at least 50%, at least 60%, at least 70%, at least 75%, at least 80%, at least 85%, at least 90% or at least 95% of these structures, e.g. as defined by in silico or in vitro screens.

In some embodiments, each second domain comprises a full regulatory element consisting of multiple RNA structures, such as a secondary structure comprising a G-quadruplex (G4), a stem loop of a multiway junction, a pseudoknot, or a combination thereof. The library may comprise multiple secondary structures, such as multiway junctions, or stem loop or G4 containing RNA constructs that comprise wildtype structures such as wild type G4, stem loop or multi way junction structure(s); and wherein other cell populations of the library comprise secondary structures such as stemloop, multiway junction, G4-containing RNA constructs that comprise mutant forms of the secondary structure such as mutated G4 structures, or mutations that disrupt multiway junction or stemloop or other structure formation. The RNA constructs in the cells may constitute a panel comprising G4 structures from a class of coding or non-coding RNA sequences expressed by an organism of interest (e.g. human), wherein the class of non-coding RNA sequences is selected from mRNA UTRs, lncRNAs, miroRNAs and cirRNAs. The RNA constructs in the cells may comprise a large proportion, or substantially all of, the G4 structures expressed by the genome of an organism of interest, for instance at least 50%, at least 60%, at least 70%, at least 75%, at least 80%, at least 85%, at least 90% or at least 95% of these structures, e.g. as defined by in silico or in vitro screens.

In some embodiments, second domain may comprise a secondary structure comprising a triple helix. The library may comprise triple helix-containing RNA constructs that comprise wildtype triple helix structure(s); and wherein other cell populations of the library comprise triple helix-containing RNA constructs that comprise mutations that can disrupt the triple helix structures. The RNA constructs in the cells may constitute a panel comprising triple helix structures from a class coding of non-coding RNA sequences expressed by an organism of interest (e.g. human), wherein the class of non-coding RNA sequences is selected from mRNA UTRs, lncRNAs, miroRNAs and cirRNAs. The RNA constructs in the cells may comprise a large proportion, or substantially all of, the triple helix structures expressed by the genome of an organism of interest, for instance at least 50%, at least 60%, at least 70%, at least 75%, at least 80%, at least 85%, at least 90% or at least 95% of these structures, e.g. as defined by in silico or in vitro screens.

In some embodiments, second domain may comprise a secondary structure comprising a pseudoknot. The library may comprise pseudoknot-containing RNA constructs that comprise wildtype pseudoknot structure(s); and wherein other cell populations of the library comprise pseudoknot-containing RNA constructs that comprise mutations that can disrupt the pseudoknot structures. The RNA constructs in the cells may constitute a panel comprising pseudoknot structures from coding and/or non-coding RNA sequences expressed by an organism of interest (e.g. human), wherein the class of non-coding RNA sequences is selected from mRNA UTRs, lncRNAs, miroRNAs and cirRNAs. The RNA constructs in the cells may comprise a large proportion, or substantially all of, the pseudoknot structures expressed by the genome of an organism of interest, for instance at least 50%, at least 60%, at least 70%, at least 75%, at least 80%, at least 85%, at least 90% or at least 95% of these structures, e.g. as defined by in silico or in vitro screens.

In some embodiments, second domain may comprise a secondary structure comprising a stem-loop. The library may comprise stem-loop-containing RNA constructs that comprise wildtype stem-loop structure(s); and wherein other cell populations of the library comprise stem-loop-containing RNA constructs that comprise mutations that can disrupt the stem-loop structures. The RNA constructs in the cells may constitute a panel comprising stem-loop structures from a class of non-coding RNA sequences expressed by an organism of interest (e.g. human), wherein the class of coding or non-coding RNA sequences is selected from mRNA

UTRs, lncRNAs, miroRNAs and cirRNAs. The RNA constructs in the cells may comprise a large proportion, or substantially all of, the stem-loop structures expressed by the genome of an organism of interest, for instance at least 50%, at least 60%, at least 70%, at least 75%, at least 80%, at least 85%, at least 90% or at least 95% of these structures, e.g. as defined by in silico or in vitro screens.

In some embodiments, each second domain of the RNA construct comprises an RNA regulatory element. In some embodiments, the RNA regulatory element is an untranslated region (UTR) or an intron from a mRNA. In some embodiments, the UTR or intron comprises a binding site that binds a long non-coding RNA (lncRNA) or an RNA-binding protein or a microRNA (miRNA). In some embodiments, the RNA each second domain comprises a 5′ UTR of a gene that is not the reporter gene. In some embodiments, the RNA each second domain comprises a 3′ UTR of a gene that is not the reporter gene.

In preferred embodiments, each second domain may comprise both a G4, stemloop, multiway junction or a combination of structures of functional structures such as an IRES. These structures/elements can be comprised within a UTR structure that forms the second domain/query sequence.

In some embodiments, the reporter gene of each first domain expresses one or more fluorescent proteins. For instance, the fluorescent protein(s) may be green fluorescent protein (GFP), blue fluorescent protein (BFP) and/or red fluorescent protein (RFP). A wide range of promoters are envisaged as being able to form part of the RNA construct, e.g. CMV, sv40, EF1a, CAG, PKG, or H1. A promoter may be present upstream of the sequences coding the reporters (e.g. GFP and RFP). Alternatively, a bidirectional promoter may be placed between them. The RNA construct may comprise an IRES of the second domain within the expression cassette, between the sequence coding for the fluorescent proteins (e.g. the sequence coding GFP and the sequence coding for RFP). A promoter may be present upstream of the sequences coding GFP and RFP (with the IRES placed between them). Alternatively, the expression cassette may comprise a coding sequence for GFP and a coding sequence for RFP, and a bidirectional promoter there-between.

In some embodiments of this invention, the second domain is positioned at the 5′ end of each RNA construct. In other embodiments, the second domain is at the 3′ end of each RNA construct.

In some embodiments, the RNA structure is positioned at the 5′ end of the reporter (ie. GFP). In other embodiments, the RNA structure is positioned at the 3′ end of the reporter.

In preferred embodiments, the RNA construct comprises a barcode sequence; and step b. further comprises a sub-step of reading the barcode sequence of the cell population(s) in which reporter gene expression is increased or decreased relative to the average reporter gene expression.

In some embodiments, step b. employs fluorescence activated cell sorting (FACS) to identify the cell population(s) in which reporter gene expression is increased or decreased relative to the average reporter gene expression, and to separate the cell populations according to reporter gene expression levels.

In some embodiments, step a. is performed by contacting the library of cells with the panel of candidate agents in a multi-well plate. In other embodiments, step a. is performed by contacting the library of cells with the panel of candidate agents via a high throughput droplet microfluidics system.

In some embodiments, the candidate agents are small molecules. In other embodiments, the candidate agents are RNA molecules, e.g. siRNAs, miRNAs or lncRNAs. Equivalent methods in which the candidate agent is a protein, e.g. RNA-binding proteins (RBPs), intrabodies, etc are also envisaged.

In some embodiments, the candidate agents are genetic perturbations introduced into patient derived samples such as an iPS derived cell line from patients with the disease. In other embodiments, the candidate agents are RNA molecules, e.g. siRNAs, miRNAs or lncRNAs. Equivalent methods in which the candidate agent is a protein, e.g. RNA-binding proteins (RBPs), intrabodies, etc are also envisaged.

In some embodiments, the screening method involves screening for agents (e.g. chemicals) or genetic perturbations that target some cell populations, but not others: The cell populations can be grouped into those that contain RNA constructs comprising elements for which targeting is desired (e.g. RNA secondary structures in cancer-related genes), and those that contain RNA constructs comprising elements for which targeting is not desired (e.g. RNA secondary structures from non-cancer-related genes). Thus, we provide a method of screening for agents that interact with multiple RNA structures, such as multiple G4, stem loop or multiway junction structures that are present in the mRNA of cancer related genes, such as MYC, RAS, VEGF, and/or KRAS, as an example. Small molecules, RNA molecules, intrabodies, etc, can be engineered to bind these subsets of RNA secondary structures simultaneously to target multiple biological processes and screened and validated using this invention.

These methods allow known and/or new agents to be identified as exhibiting RNA-targeting modes of action.

In a further aspect, this invention provides a method of producing a library of cells by transfecting or transducing a population of cells with the library of vectors described herein. The cells may be mammalian, e.g. human cells. The cells may be stem cells, e.g. induced pluripotent stem cells (IPS cells). The cells may be tumor cells. The cells may be from a tissue sample. The cells may be from a biopsy from a mammalian subject, e.g. a human subject.

In a further aspect, this invention provides a multiplexed method of screening a panel of candidate agents to select agents that interact with an RNA regulatory element and/or secondary structure, the method comprising: a. contacting the library of cells with the panel of candidate agents in a multiplexed fashion, then b. identifying one or more cell populations in which reporter gene expression is increased or decreased relative to the average reporter gene expression, and then c. selecting the candidate agents that had been contacted with said cell populations identified in step b.

The RNA constructs may comprise a barcode sequence and step b. may further comprise reading the barcode sequence of any of the cell populations in which reporter gene expression is increased or decreased relative to the average reporter gene expression.

Step a. may be performed by contacting the library of cells with the panel of candidate agents in a multi-well plate, or by contacting the library of cells with the panel of candidate agents via a high throughput droplet microfluidics system. In some embodiments, the candidate agents are small molecules or RNA molecules such as siRNA, miRNA or lncRNA.

Step b. may employ fluorescence activated cell sorting (FACS) to identify the cell population(s) in which reporter gene expression is increased or decreased relative to the average reporter gene expression, and to separate the cell populations according to reporter gene expression levels.

In a further aspect, this invention provides a method of screening a population of RNA regulatory elements and/or secondary structures to assess the function of said element/structure, the method comprising providing a library of cells, propagating the cells, and assessing the expression level of the reporter gene in each cell population. Each RNA regulatory element may be a UTR. Each RNA secondary structure may be a G4. The RNA constructs may comprise all G4 structures expressed by the genome of an organism of interest. The RNA constructs may constitute a panel comprising G4 structures from a class of coding or non-coding RNA sequences expressed by an organism of interest, wherein the class of non-coding RNA sequences is selected from introns or UTRs of mRNA, lncRNA, miroRNA or cirRNA. Each RNA regulatory element and/or secondary structure may comprise an internal ribosome entry site (IRES). The RNA constructs may comprise all IRES structures expressed by the genome of an organism of interest. The RNA constructs may constitute a panel comprising IRES structures from a class of coding or non-coding RNA sequences expressed by an organism of interest, wherein the class of non-coding RNA sequences is selected from introns or UTRs of mRNA, lncRNA, miroRNA or cirRNA. Each RNA regulatory element and/or secondary structure comprises a triple helix. The RNA constructs may comprise all triple helix structures expressed by the genome of an organism of interest. The RNA constructs may constitute a panel comprising triple helix structures from a class of coding or non-coding RNA sequences expressed by an organism of interest, wherein the class of non-coding RNA sequences is selected from introns or UTRs of mRNA, lncRNA, miroRNA or cirRNA. Each RNA regulatory element and/or secondary structure comprises a pseudoknot. The RNA constructs comprise all pseudoknot structures expressed by the genome of an organism of interest. The RNA constructs may constitute a panel comprising pseudoknot structures from a class of coding or non-coding RNA sequences expressed by an organism of interest, wherein the class of non-coding RNA sequences is selected from introns or UTRs of mRNA, lncRNA, miroRNA or cirRNA.

In some embodiments, the cells have been genetically modified to knock-out, knock-down, or silence one or more RNA-binding proteins.

In a further aspect, this invention provides a method of identifying genetic perturbations associated with a disease condition, the method comprising providing a library of cells comprising the nucleic acid constructs of the invention, wherein a first subpopulation of the cells comprises RNA regulatory elements and/or secondary structures from one or more subjects having a particular disease; and a second subpopulation of the cells comprises RNA regulatory elements and/or secondary structures from one or more subjects that do not a particular disease; and comparing the relative reporter gene expression levels in the first and second subpopulations

In a further aspect, this invention provides a method of identifying chemical perturbations associated with a disease condition, the method comprising providing a library of cells comprising the nucleic acid constructs of the invention, and contacting a first subpopulation of the cells with one or more chemical agents; and comparing the relative reporter gene expression levels in the first subpopulation with that of a second subpopulation that has not been contacted with the one or more chemical agents, e.g. small molecules, known therapeutic agents, and/or further RNA molecules.

In a further aspect, this invention provides a method of creating a cellular network or biological phenotype or circuit, the method comprising (a) introducing at least one, two, three, four or more single-order or combinatorial genetic perturbations to the query sequence in a library of cells comprising the nucleic acid constructs of the invention, wherein each cell in the plurality of the cells receives at least one perturbation; (b) a measuring process comprising: (i) detecting genomic, genetic, proteomic, epigenetic and/or phenotypic differences in single cells compared to one or more cells that did not receive any perturbation, and (ii) detecting the perturbation(s) in single cells; and (c) determining measured differences relevant to the perturbations by applying a model accounting for co-variates to the measured differences, whereby intercellular and/or intracellular networks or circuits are inferred. The measuring process (b) may comprise single cell sequencing and/or reading a barcode sequence that may be present in the nucleic acid construct. The model may account for the capture rate of measured signals, whether the perturbation actually perturbed the cell (phenotypic impact), the presence of subpopulations of either different cells or cell states, and/or analysis of matched cells without any perturbation.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows three diagrammatic representations of the reporter construct utilised to investigate the function of RNA structures. In FIG. 1A, the sequence of an RNA structure or its mutated version are cloned immediately upstream of d2EGFP. mCherry is expressed by its own promoter and serves as an internal control. Each reporter, representing a different RNA structure being investigated contains a unique barcode cloned immediately downstream of the EF1a promoter. In FIG. 1B, the RNA structure is cloned immediately downstream of the d2EGFP. Each reporter, representing a different RNA structure being investigated contains a unique barcode cloned downstream of the EF1a promoter. In FIG. 1C, a bicistronic reporter system is represented. The EF1a promoter is upstream of two reporter genes (d2GFP and mCherry) which flank an IRES. A sequence which will form a hairpin is cloned immediately downstream of the d2GFP which serves to prevent ribosome readthrough. Each reporter, representing a different IRES element being investigated contains a unique barcode cloned immediately upstream of the IRES. Puromycin is expressed by its own promoter hPGK and serves as an internal control.

FIG. 2 shows a general overview of the assay workflow. Multiple reporters with distinct RNA structures are first generated and packaged into lentiviruses. Lentiviruses are pooled and MOI of the pool is determined. Cells are infected at a low MOI to ensure single reporter integration per cell. Infected cells untreated, or treated with small molecules which modulate the RNA structure are FACS sorted on their basis of mCherry and GFP expression. Sorted populations are then subjected to PCR amplification of the barcode and NGS.

FIG. 3 shows an example of an amplicon sequencing strategy. (A) Universal forward and reverse primers were designed in the constant regions flanking the unique barcode to yield an amplicon product of 200-250nt. (B) A 2-step PCR strategy is employed; 1. Region with embedded barcode is amplified from gDNA using described primers. 2. Forward and reverse primers are annealed onto the product of the first PCR. Both forward and reverse primers carry unique sample indexes. (C) gDNA was isolated from cell pools infected with reporters, and the barcode region was amplified using PCR. PCR product analysed on Agilent TapeStation and reveals appropriate insert size. In different embodiments, primers were designed to amplify the barcode of structures cloned in the 3′UTR of d2EGFP.

FIG. 4 shows an example flow cytometry analysis workflow for an exemplary target LTX032, which is a G quadruplex (G4). (A) Typical flow cytometry gating strategy employed to interrogate GFP expression in cells infected with reporter lentivector. Gates were set to identify single, live cells. GFP expression was then quantified in mCherry positive cells. (B) Histograms representing GFP expression in cells infected with 3 different reporter constructs; 1. 5′UTR empty vector, 2. LTX032 (RNA structure) cloned into 5′UTR, 3. LTX032sc (mutated version of the same RNA structure) cloned into the cassette upstream of the reporter.

FIG. 5 shows an example of testing RNA structure functionality in reporter assay. Cell pools were infected with individual reporter constructs at a constant MOI. Infection efficiency and mCherry expression level, determined by mCherry % cells and mCherry MFI respectively, were constant across the infected cell populations. Reduction in GFP expression was observed in cell pools infected with reporter containing the wild-type RNA structure, relative to the mutated control and empty vector. This indicates that this example RNA structure is functional in suppressing the translation of GFP.

FIG. 6 shows an example of pooled lentiviral approach. Lentiviruses for 10 reporters, including wild-type RNA structures and their mutant versions as well as empty vectors, were manually pooled and the MOI of the pool was determined. (A) Cells were infected with the lentiviral pool and subjected to flow cytometric analysis. Gates were set to identify single, live cells. GFP expression distribution was quantified in mCherry positive cells. Cells were sorted on the basis of their GFP expression. Sorted cell populations were subjected gDNA extraction and PCR amplification of the barcodes utilising the universal primers, as described in FIG. 3. Amplified products were subjected to NGS. (B) Reporters were deconvolved on the basis of their barcode, and fold change was computed between GFP low/GFP low cells.

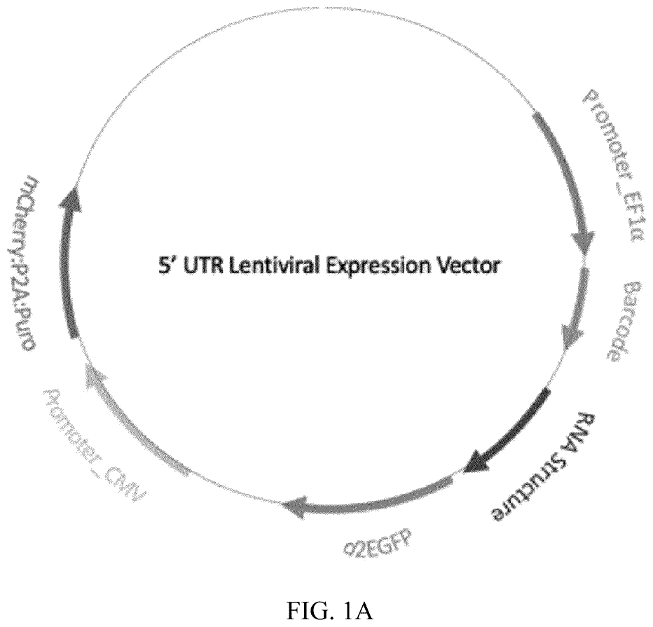

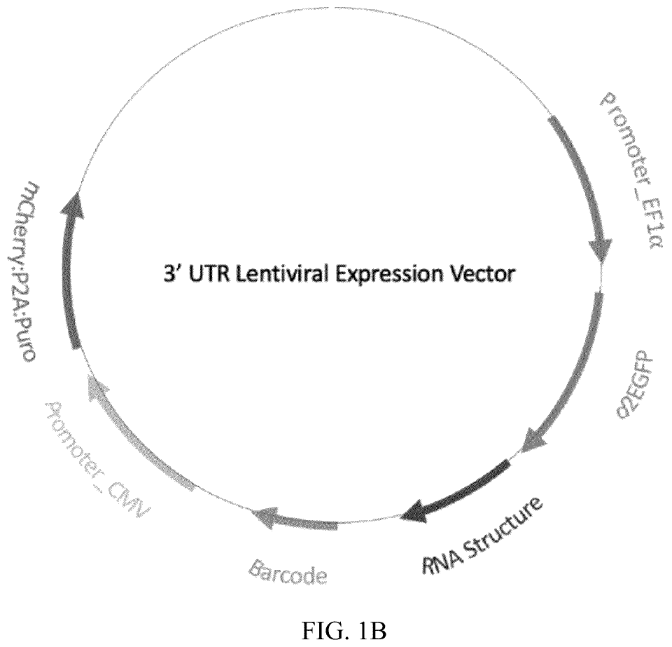

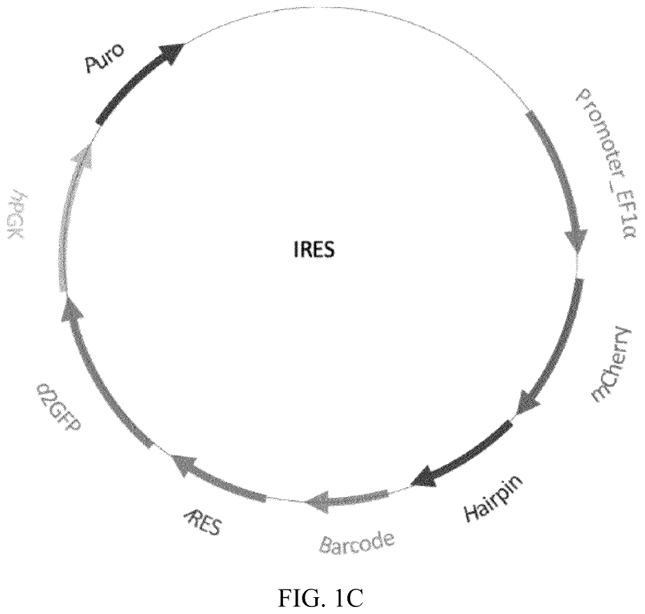

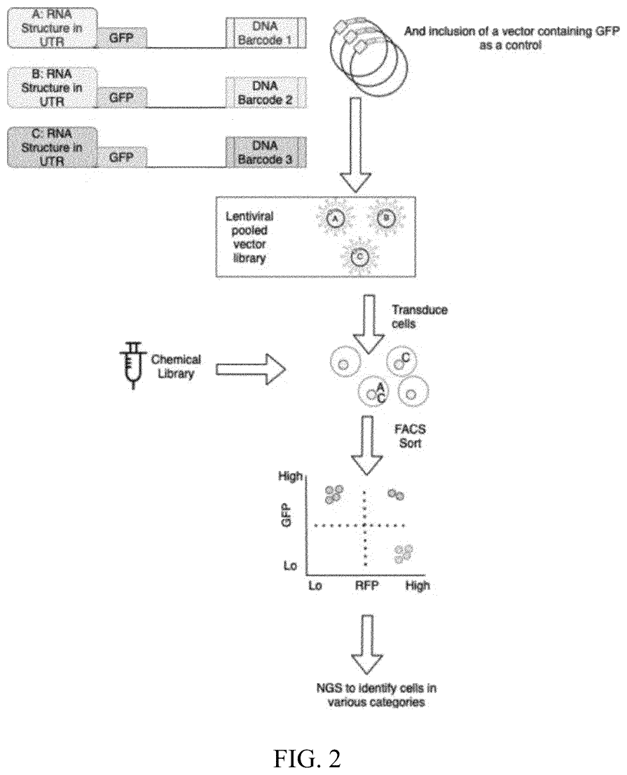

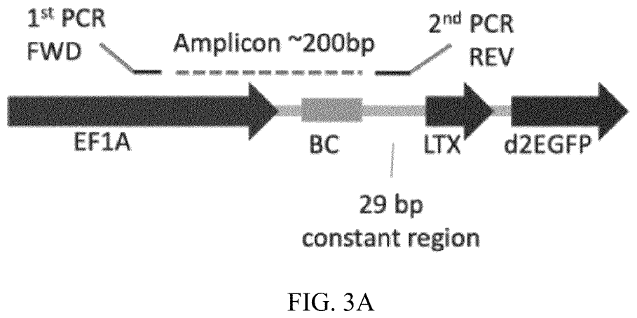

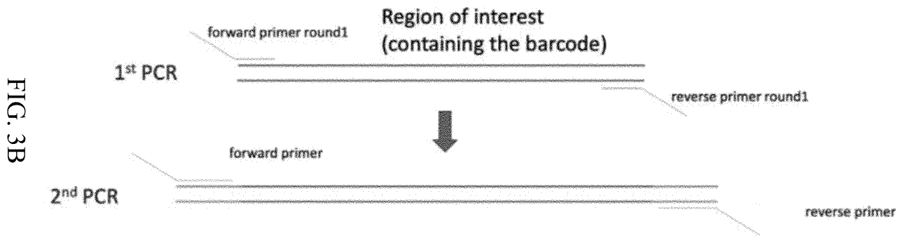

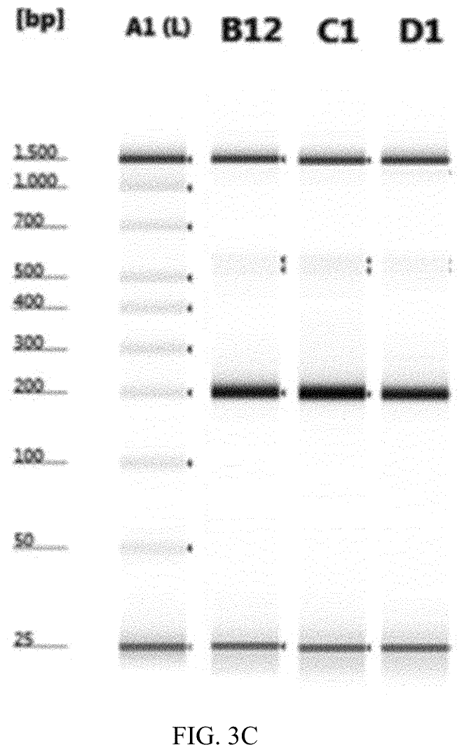

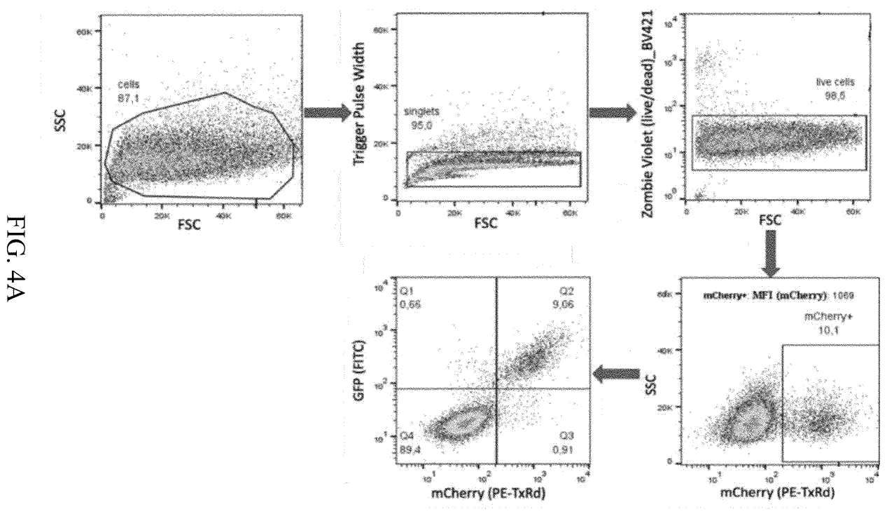

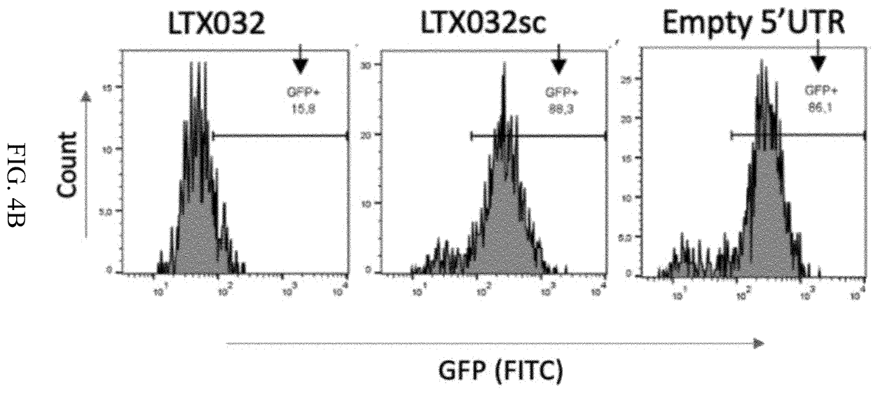

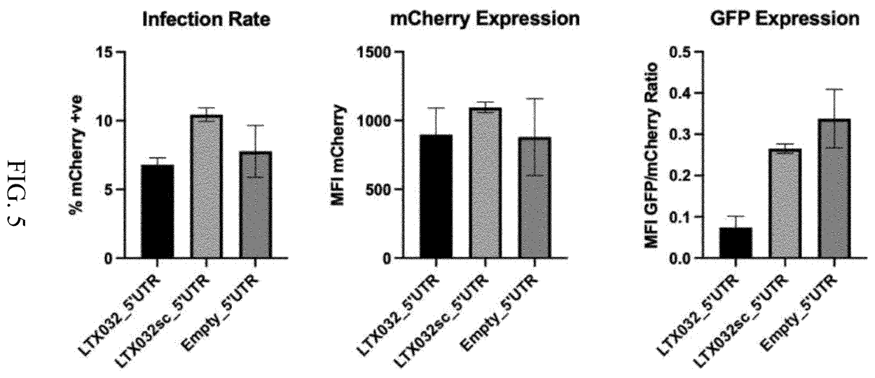

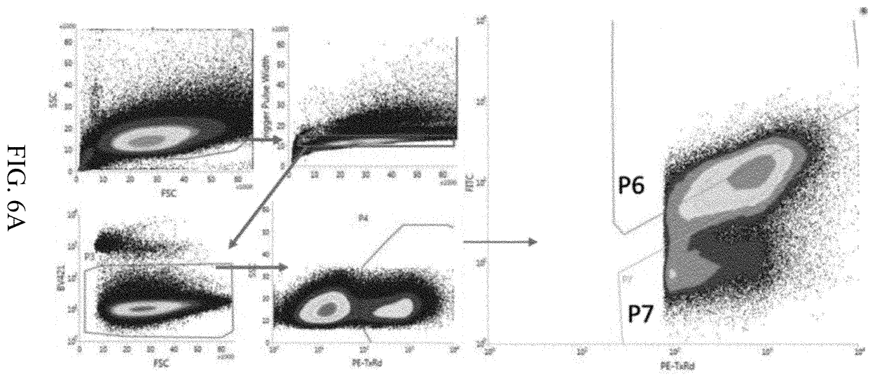

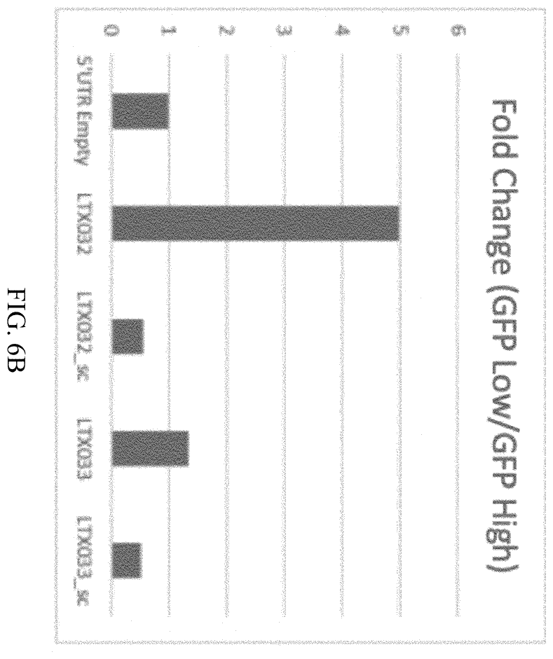

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Herein, we describe a cellular-screening platform that enables the screening of genetic or chemical libraries against multiple RNA structures within a specific RNA structural class (such as G-quadruplexes, Triple Helix, Pseudoknots, multiway junctions, or the combination thereof in a functional element such as an Internal Ribosomal Entry Site (IRES).

Vectors may be used to express the RNA structures in the eukaryotic cell. Vectors that may be used include viral vectors, plasmids, cosmids and artificial chromosomes. Viral vectors that may be used include retrovirus vectors, adenovirus vectors and adeno-associated viruses (AAV). Retrovirus vectors include lentiviral vectors. The vectors of the invention may lead to stably-transfected cells or transiently transfected cells.

In one embodiment, the genetic perturbations of the RNA structural motif can have an impact on the expression of a reporter gene. In some embodiments, this reporter gene expresses one or more fluorescent proteins. The altered expression of the reporter gene leads to an altered fluorescent readout, and this fluorescent readout is used to separate the cells using a cell-sorting mechanism. The cells within each category are then sorted and a DNA barcode on the RNA constructs is then used to sequence the cells within each pool to enable easy mapping of the original constructs within each category.

In one embodiment, the binding of a small molecule to the RNA structural motif can have an impact on the expression of a reporter gene. In some embodiments, this reporter gene expresses one or more fluorescent proteins. The altered expression of the reporter gene leads to an altered fluorescent readout, and this fluorescent readout is used to separate the cells using a cell-sorting mechanism. The cells within each category are then sorted and a DNA barcode on the RNA constructs is then used to sequence the cells within each pool to enable easy mapping of the original constructs within each category.

Thus, a number of different functional RNA structures such as G-quadruplexes, triple helices, pseudoknots, and multiway junctions are amenable to investigation, or the combination thereof, which can form a function structure such as an IRES element (internal ribosomal entry site). This approach enables the study of the effects of multiple genetic perturbations in parallel or the effect of compound sets on multiple similar RNA structures in parallel. This approach allows us to identify the effect of genetic mutations or chemical perturbations at scale. This will allow generic RNA-motif binders and binders which specifically bind to particular RNA structure to be distinguished from each other. Furthermore, this approach can also be applied to medicinal chemistry campaigns to screen chemical compounds from the scientific literature and study the effects of chemical modifications on specificity and selectivity across a given RNA-structural class, such as G-quadruplexes, stem loops or multiway junctions. The combination of these can form functional elements such as internal ribosomal entry sites, or binding sites for splicing sites for RNA binding proteins such as HNRNP.

Similar to kinase panels, an RNA panel, such as a G-quadruplex or IRES panel may be assembled. These types of RNA secondary structures are briefly discussed in the sections below.

### G-Quadruplex (G4)

A G-quadruplex (G4) is a nucleic acid tertiary structure, formed by the association of guanine bases, either on the same or different strands. Four guanine bases interact by Hoogsteen hydrogen bonding to form a guanine tetrad (G-tetrad), a square planar structure that is capable of hydrogen bonding with other G-tetrads to form a G-quadruplex. G4 are thought to be involved in a wide array of biological functions, such as transcriptional regulation, telomere length regulation and immunoglobulin heavy chain switching.

### Internal Ribosome Entry Site (IRES)

An IRES is an RNA element that coordinates alternative translation by triggering translation initiation in a cap-independent manner. An IRES may be located in the 5′UTR or elsewhere in the RNA. 10% of mammalian mRNA have an IRES element that can be used for alternative translation. The structure of IRES are important for their function, as the ribosome is recruited to the IRES by binding to its secondary or tertiary structure.

### Dual Promoter Reporter Systems

Translational control of an RNA structure can be measured using a dual promoter reporter system. This reporter comprises an RNA structure localised in the 5′ or 3′ end of an ORF. The ORF codes for a reporter gene, such as firefly luciferase or GFP. Translation of the ORF is then regulated by this RNA structure. If the structure functions to repress translation, then a loss of fluorescent signal is anticipated. Conversely, if the structure functions to enhance translation, then a gain of fluorescent signal is anticipated. A second promoter is included to drive expression of a second ORF. The second ORF codes for a different reporter gene, such as RFP and renilla luciferase, which serves as an internal control.

### Bicistronic Reporter Systems

IRES activity can be measured by bicistronic reporter systems. This comprises an mRNA with a promoter upstream of two complete open reading frames (ORFs) flanking an IRES. Each ORF codes for a reporter gene, such as GFP or firefly luciferase. Upon transcription of the mRNA, the ORF upstream of the IRES is translated in a cap-dependent manner, whilst the ORF downstream of the IRES is translated in a cap-independent manner, initiated by the IRES.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organisational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### Definitions

In this specification the term “operably linked” may include the situation where a selected nucleotide sequence and regulatory nucleotide sequence are covalently linked in such a way as to place the expression of a nucleotide coding sequence under the influence or control of the regulatory sequence. Thus a regulatory sequence is operably linked to a selected nucleotide sequence if the regulatory sequence is capable of effecting transcription of a nucleotide coding sequence which forms part or all of the selected nucleotide sequence. Where appropriate, the resulting transcript may then be translated into a desired protein or polypeptide.

Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

Where the method is performed in vitro it may comprise a high throughput screening assay. Test compounds used in the method may be obtained from a synthetic combinatorial peptide library, or may be synthetic peptides or peptide mimetic molecules. Other test compounds may comprise defined chemical entities, oligonucleotides or nucleic acid ligands.

### EXAMPLES

### Example 1

### Design of Reporter Amenable to High Throughput Testing for Functional Interrogation of RNA Structures

Lentivector backbone was modified to include the following features; destabilised GFP (d2EGFP), mCherry:Puromycin fusion product which serves as an internal control for transfection, and a barcode. In this reporter, the EF1a promoter drives expression of d2GFP. This insert carrying the RNA structures, either wild type or mutant, was cloned into the UTR of d2EGFP in the lentivector, to study its functional effect on translation of d2EGFP. Here, mCherry is expressed independently under the control of a CMV promoter and serves as an internal control. For each RNA structure investigated, lentivectors were also generated which contained a mutated version of the RNA structure. Lentivectors were packaged and lentivirus titer was measured using p24 ELISA method.

### Example 2

### Functional Assessment of RNA Structures Using Reporter System

Lentiviral infection was optimised first. 4,000 cells per well were infected with 3 different MOls (3, 10, 30). Infection was performed with or without polybrene (2, 4, or 8 ug/mL), and with or without spinfection (900 g for 1 hour). Fluorescence was assessed in the various infection conditions 48 hours post infection using IncuCyte. 8 ug/mL of polybrene in combination with spinfection resulted in highest infection rates, however infection was efficient in the absence of polybrene or spinfection. No significant effect on cell proliferation due to infection, polybrene or spinfection was observed. To assess expression dynamics of the fluorescent proteins, cells were infected as above and fluorescence was monitored using IncuCyte. Both d2GFP and mCherry became detectable between 16-20 hours after infection. d2GFP signal reached plateau after 32-36 hours due to the destabilised nature.

200,000 cells per well (in 6-well format) were infected at MOI of 1, without polybrene, or spinfection. Virus containing medium was removed after 18 hours and replaced by fresh medium. Cells were cultured for an additional 24 hours. Forty-two hours post infection, cells were washed with PBS, detached and transferred to 96-well format for viability staining. Cells were stained with Zombie Violet Fixable Viability dye. Zombie Violet dye was diluted at 1:1000 in PBS (18 ml+18 μl ZoVi) and 100 uL was added to each well. Cell solution was incubated in the dark for 20 minutes at RT. Cells were spun down and supernatant was removed. Cells were washed one time with 150 μl BioLegend's Cell Staining Buffer (Cat. No. 420201), and supernatant was removed following a spin down. 100 uL of 4% PFA solution was added to each well, and mixed immediately to avoid clustering. Cells were fixed in the dark for 20 minutes at RT. Following spin down, cells were washed with 100 uL PBS. Stained and fixed cells were stored dark at 4° C. until acquisition with the FACSJazz instrument.

### Example 3

### Pooled Lentiviral Transduction

Lentiviral pool was generated by pooling 10 lentiviruses containing either WT, MUT or control lentivectors. Titer of the pool was determined to be 3.4×10\hat{ }8 TU/ml. 20 million cells were analyzed using FACS. Infection rate of the pool was determined to be 29.2%. 100,000 cells in “GFP high” category (top 25%) and 72,000 cells in “GFP low” category (bottom 25%) were obtained following cell sorting.

### Example 4

gDNA Isolation, PCR and Sequencing

DNA isolation was performed using the GeneRead FFPE kit. Total of 344 ng of DNA was isolated from “GFP high” sorted population and 305.12 ng was isolated from “GFP low” sorted populations. 100 ng input material was used for the PCR reaction. Forward and reverse primers, 21nt in length were designed to amplify the barcode region such that a 200nt amplicon would be generated. Tm of primers was 61 degrees, and GC content was 57%. Forward and reverse primers were then annealed onto the product of the first PCR reaction. Both forward and reverse primers carry unique sample indexes. PCR product was analyzed on Agilent TapeStation.

### REFERENCES

A number of publications are cited above in order to more fully describe and disclose the invention and the state of the art to which the invention pertains. Full citations for these references are provided below. The entirety of each of these references is incorporated herein.
Jones et al., “A Scalable, Multiplexed Assay for Decoding GPCR-Ligand Interactions with RNA Sequencing”, 2019, Cell Systems 8, 254-260
EP3649236A1, “Multiplexed receptor-ligand interaction screens” (Kosuri & Jones)
Dixit et al., “Perturb-seq: Dissecting molecular circuits with scalable single cell RNA profiling of pooled genetic screens” Cell. 2016; 167(7): 1853-1866.e17.
WO 2017/075294, “Assays For Massively Combinatorial Perturbation Profiling And Cellular Circuit Reconstruction” (Regev et al.)
Rizvi et al., “RNA-ALIS: Methodology for screening soluble RNAs as small molecule targets using ALIS affinity-selection mass spectrometry”, Methods 2019; 167:28-38
Fardokht et al., “Selective Small-Molecule Targeting of a Triple Helix Encoded by the Long Noncoding RNA, MALAT1”, CS Chem. Biol. 2019, 14, 2, 223-235
Connelly et al., “Discovery of RNA Binding Small Molecules Using Small Molecule Microarrays”, Methods Mol Biol. 2017; 1518: 157-175.
Pedram Fatemi et al., “Screening for Small-Molecule Modulators of Long Noncoding RNA-Protein Interactions Using AlphaScreen”, J Biomol Screen. 2015 October; 20(9):1132-41
Lorenz et al., “Development and Implementation of an HTS-Compatible Assay for the Discovery of Selective Small-Molecule Ligands for Pre-microRNAs”, SLAS Discov. 2018 January; 23(1): 47-54.
Sidarovich et al., “A Cell-Based High-Throughput Screen Addressing 3′UTR-Dependent Regulation of the MYCN Gene”, Mol Biotechnol. 2014; 56(7): 631-643.
Yang et al., “IRES-mediated cap-independent translation, a path leading to hidden proteome”, J Mol Cell Biol. 2019 Oct. 25; 11(10):911-919
Hejazi Pastor, “Targeting the CACNA1A IRES as a Treatment for Spinocerebellar Ataxia Type 6”, Cerebellum. 2018 February; 17(1):72-77
Vaklavas et al., “Small molecule inhibitors of IRES-mediated translation”, Cancer Biol Ther. 2015; 16(10):1471-85

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. A multiplexed method of screening a panel of candidate agents to select agents that interact with an RNA regulatory element and/or secondary structure, the method comprising:
a. contacting a library of cells with the panel of candidate agents in a multiplexed fashion, wherein the library of cells comprises a multiplicity of cell populations each comprising one or more cells comprising an RNA construct, wherein each RNA construct of the cells of one single population are the same as each other, but wherein the RNA constructs of cells of different populations are different from each other, wherein each RNA construct comprises a first domain comprising an expression cassette capable of expressing a reporter gene, and a second domain in which the RNA is folded into a secondary structure or comprises an RNA regulatory element of a gene transcript that is not the reporter gene transcript,
b. identifying a cell population in which reporter gene expression is increased or decreased relative to the average reporter gene expression, and
c. selecting the candidate agents that had been contacted with said cell population identified in step (b).

121. The multiplexed method according to claim 120, wherein the RNA constructs comprise a barcode sequence and wherein step b. further comprises reading the barcode sequence of the cell population(s) in which reporter gene expression is increased or decreased relative to the average reporter gene expression.

122. The multiplexed method of claim 120, wherein step b. employs fluorescence activated cell sorting (FACS) to identify the cell population(s) in which reporter gene expression is increased or decreased relative to the average reporter gene expression, and to separate the cell populations according to reporter gene expression levels.

123. (canceled)

124. (canceled)

125. The multiplexed method according to claim 120, wherein the candidate agents are small molecules.

126. The multiplexed method according to claim 120, wherein the candidate agents are RNA molecules.

127. (canceled)

128. (canceled)

129. (canceled)

130. The method according to claim 120, wherein each RNA regulatory element or secondary structure comprises a G-quadruplex (G4), a triple helix, a pseudoknot, a stem-loop, or a multiway junction.

131. (canceled)

132. The method according to claim 130, wherein the RNA constructs comprise a panel of G4 structures or all G4 structures expressed by the genome of an organism of interest.

133. The method according to claim 130, wherein each RNA regulatory element or secondary structure comprises an internal ribosome entry site (IRES).

134. (canceled)

135. The method according to claim 133, wherein the RNA constructs comprise all IRES structures expressed by the genome of an organism of interest.

136. (canceled)

137. (canceled)

138. The method according to claim 130, wherein the RNA constructs comprise a panel of triple helix structures, or all triple helix structures expressed by the genome of an organism of interest.

139. (canceled)

140. (canceled)

141. The method according to claim 130, wherein the RNA constructs comprise a panel of pseudoknot structures or all pseudoknot structures expressed by the genome of an organism of interest.

142. (canceled)

143. (canceled)

144. (canceled)

145. (canceled)

146. (canceled)

147. (canceled)

148. (canceled)

149. (canceled)

150. (canceled)

151. The method according to claim 130, wherein the RNA constructs comprise a panel of stem-loop structures or all stem-loop structures expressed by the genome of an organism of interest.

152. The method according to claim 130, wherein the RNA constructs comprise a panel of multiway junction structures or all multiway junction structures expressed by the genome of an organism of interest.

153. The method of claim 120, wherein step (b) includes identifying the secondary structures of the RNA constructs from the cell populations in which reporter gene expression is increased or decreased by the candidate agent identified in step (c).

154. The method of claim 130, wherein step (b) includes identifying the secondary structures of the RNA constructs from the cell populations in which reporter gene expression is increased or decreased by the candidate agent identified in step (c).

155. The method of claim 132, wherein step (b) includes identifying the G4 structures of the RNA constructs from the cell populations in which reporter gene expression is increased or decreased by the candidate agent identified in step (c).

156. The method of claim 138, wherein step (b) includes identifying the triple helix structures of the RNA constructs from the cell populations in which reporter gene expression is increased or decreased by the candidate agent identified in step (c).

157. The method of claim 141, wherein step (b) includes identifying the pseudoknot structures of the RNA constructs from the cell populations in which reporter gene expression is increased or decreased by the candidate agent identified in step (c).

158. The method of claim 151, wherein step (b) includes identifying the helix-loop structures of the RNA constructs from the cell populations in which reporter gene expression is increased or decreased by the candidate agent identified in step (c).

159. The method of claim 152, wherein step (b) includes identifying the multiway junction structures of the RNA constructs from the cell populations in which reporter gene expression is increased or decreased by the candidate agent identified in step (c).
